(19) United States
(12) Patent Application Publication
(10) Pub. No.: US 2026/0347761 A1
(43) Pub. Date: Nov. 5, 2026

(54) NEW BIFIDOBACTERIUM ADOLESCENTIS

(71) Applicant: KABUSHIKI KAISHA YAKULT HONSHA, Tokyo (JP)

(72) Inventors: Toru ODANI, Tokyo (JP); Yusuke NAGARA, Tokyo (JP); Daichi FUJII, Tokyo (JP); Kenji OISHI, Tokyo (JP)

(73) Assignee: KABUSHIKI KAISHA YAKULT HONSHA, Tokyo (JP)

(21) Appl. No.: 19/148,392

(22) PCT Filed: Jan. 31, 2023
(86) PCT No.: PCT/JP2023/003018

§ 371 Date: Jul. 16, 2025

(51) Int. Cl. C12N 1/205; C12R 1/01

(52) U.S. Cl. CPC C12N 1/205; C12R 2001/01

(57) ABSTRACT

Using Bifidobacterium adolescentis which has a function of adhering to and utilizing a starch granule and where a culture supernatant thereof has a property of suppressing adhesion of another bacterium to a starch granule, Bifidobacterium adolescentis which is expected to exert generally known advantages of bifidobacteria, such as the metabolism-improving effect through acetic acid, suppression of an allergy, protection against infection and enhancement of the effect of an anticancer agent, can be proliferated conveniently when starch is taken.

### TECHNICAL FIELD

The present invention relates to Bifidobacterium adolescentis which has a property of suppressing adhesion of another bacterium to a starch granule.

### BACKGROUND ART

When Bifidobacterium adolescentis, which is a species of bifidobacteria, is taken, generally known advantages of bifidobacteria, such as the metabolism-improving effect through acetic acid, suppression of an allergy, protection against infection and enhancement of the effect of an anticancer agent, are expected to be obtained (NPL 1).

Here, when a human takes starch, intestinal microbiota adhere to and colonize on the starch in the intestines, and thus the microbiota composition composition and the production state of metabolites and the like change. Known examples of such intestinal microbiota which adhere to and colonize on starch include Eubacterium rectale, Bifidobacterium adolescentis and the like.

These intestinal microbiota, however, are in competition (NPL 2), and thus it has been impossible so far to proliferate Bifidobacterium adolescentis alone conveniently when starch is taken.

### CITATION LIST

### Non Patent Literature

NPL 1: Amy O'Callaghan, Douwe van Sinderen, Bifidobacteria and Their Role as Members of the Human Gut Microbiota, Front Microbiol. 2016; 7:925. (doi:10.3389/fmicb.2016.00925)

NPL 2: Ines Martinez, Jaehyoung Kim, Patrick R. Duffy, Vicki L. Schlegel, and Jens Walter, Resistant Starches Types 2 and 4 Have Differential Effects on the Composition of the Fecal Microbiota in Human Subjects, PLoS One. 2010; 5(11):e15046. (doi:10.1371/journal.pone.0015046)

### SUMMARY OF INVENTION

### Technical Problem

An object of the invention is to provide a technique for proliferating Bifidobacterium adolescentis which is expected to exert the above advantages conveniently when starch is taken.

### Solution to Problem

As a result of extensive studies to solve the problem, the present inventors have found that Bifidobacterium adolescentis having a specific property suppresses adhesion of intestinal microbiota in competition such as Eubacterium rectale to starch granules and adheres and colonizes itself, and the invention has been thus completed.

That is, the invention relates to Bifidobacterium adolescentis which has a function of adhering to and utilizing a starch granule and which is characterized in that a culture supernatant thereof has a property of suppressing adhesion of another bacterium to a starch granule.

Moreover, the invention relates to Bifidobacterium adolescentis YIT 13021 (NITE ABP-03808), Bifidobacterium adolescentis YIT 13022 (NITE ABP-03809), Bifidobacterium adolescentis YIT 11033 (NITE ABP-03810), Bifidobacterium adolescentis YIT 11034 (NITE ABP-03811) and Bifidobacterium adolescentis YIT 11040 (NITE ABP-03812).

Furthermore, the invention relates to a composition containing the Bifidobacterium adolescentis and a starch granule.

In addition, the invention relates to a culture supernatant of the Bifidobacterium adolescentis.

### Advantageous Effects of Invention

When the Bifidobacterium adolescentis of the invention is taken with the intake of starch, the Bifidobacterium adolescentis is expected to suppress the proliferation of Eubacterium rectale, which is believed to proliferate in the intestines efficiently through adhesion to starch granules, and to proliferate mainly instead.

As a result, after intake of starch granules, Bifidobacterium adolescentis becomes the mainly proliferating species in all the people who have taken Bifidobacterium adolescentis, and generally known effects of bifidobacteria which are exerted by the proliferation of Bifidobacterium adolescentis, such as the metabolism-improving effect through acetic acid, suppression of an allergy, protection against infection and enhancement of the effect of an anticancer agent, are expected to be exerted.

### BRIEF DESCRIPTION OF DRAWINGS

FIG. 1 A figure showing Eubacterium rectale strain G38colonizing on pSt (potato starch granules) (ABC: under the condition without the addition of a culture supernatant of strain YIT 13021, and DEF: under the condition with the addition of a culture supernatant of strain YIT 13021).

FIG. 2 A figure showing the change in the pSt-adhering bacterial cell amount of strain G38 with the addition of a culture supernatant of strain YIT 13021 (the dots show the luminance values of the respective granules, and the box plot shows the quartiles of the respective culture conditions).

FIG. 3 A figure showing the bright field images taken after adding equivalent amounts of the indicated culture supernatants to a culture solution of strain G38 colonizing on pSt and stirring at 37 °C for five hours.

FIG. 4 A figure showing the bright field images taken after adding equivalent amounts of the indicated culture supernatants to a culture solution of strain G38 colonizing on pSt and stirring at 37 °C for five hours.

### DESCRIPTION OF EMBODIMENTS

The Bifidobacterium adolescentis of the invention is Bifidobacterium adolescentis which has a function of adhering to and utilizing a starch granule, and a culture supernatant thereof has a property of suppressing adhesion of another bacterium to a starch granule. Here, a culture supernatant of the type strain of Bifidobacterium adolescentis (ATCC15703 ) does not have any property of suppressing adhesion of another bacterium to a starch granule.

The Bifidobacterium adolescentis of the invention can be obtained as follows.

First, Bifidobacterium adolescentis is selected from a sample of the stool, the colon content or the like of a mammal such as a human according to a general method. Examples of the general method include the methods described in (1) Study of Bifidobacteria, edited and written by Tomotari Mitsuoka, The Japan Bifidus Foundation, (2) Comparison of intestinal bacteria composition identified by various analytical methods, Tomohiko Fujisawa, Japanese Journal of Bacteriology, 2014, (3) PMID: 28394924 (although Bifidobacterium adolescentis is isolated from mouse stool, the method can be applied to human) and the like.

It is examined that the selected Bifidobacterium adolescentis has a function of adhering to and utilizing a starch granule and that a culture supernatant thereof has a property of suppressing adhesion of another bacterium to a starch granule.

Examples of the method for examining that the Bifidobacterium adolescentis has a function of adhering to and utilizing a starch granule include a method of examining that the Bifidobacterium adolescentis has a function of adhering to a starch granule of potato starch or the like which has not been heated in a water-containing state such as autoclaving and a method of examining that the Bifidobacterium adolescentis has a function of utilizing the starch.

Here, the starch granules refer to those of starch derived from tubers such as potatoes and sweet potatoes, grains such as wheat, rice and corn, beans such as soybeans and adzuki beans, tapioca, bananas or the like which have not undergone a heating step containing water (boiling, steaming or the like) except for the drying step during the production or which have not been gelatinized and which maintain the natural granular state and are insoluble in water at normal temperature even when the starch granules have undergone a heating step containing water. More specifically, the starch granules are those of uncooked starch derived from uncooked tubers such as potatoes and sweet potatoes, grains such as wheat, rice and corn, beans such as soybeans and adzuki beans, tapioca, bananas or the like which have a granular shape such as sphere, egg shape and a polyhedron, and the diameter thereof is typically 1 to 120 μm although the diameter depends on the plant species. The diameter can be measured using a microscope or a particle diameter analyzer.

Specific methods of the above methods are the methods described in (2) Evaluation of Property of Adhering to Starch Granules and (3) Evaluation of Property of Utilizing Starch Granules in Example 4. In the evaluation method of (2), it is determined that the strain has a function of adhering to a starch granule when aggregates of starch granules and the bacterial cells are observed visually or under a microscope. Moreover, in the evaluation method of (3), it is determined that the strain has a function of utilizing a starch granule when the pH of the culture solution becomes less than 5.

An example of the method for examining that a culture supernatant has a property of suppressing adhesion of another bacterium to a starch granule is a method of examining that a culture supernatant of a one-day culture using a culture medium such as GAM medium or the like has a property of suppressing adhesion of another bacterium to a starch granule.

Here, the culture supernatant refers to a supernatant obtained after culturing Bifidobacterium adolescentis in a culture medium such as GAM medium and then centrifuging the culture.

Here, the other bacterium is not particularly limited but is, for example, a bacterium selected from the group consisting of Ruminococcus bromii, Collinsella aerofaciens and Eubacterium rectale, preferably Eubacterium rectale.

A specific method of the above method is the method described in (1) to (5) in Example 1. In the method, it is determined that the culture supernatant has a property of suppressing adhesion of another bacterium to a starch granule when the luminance value of Hoechst 33342 as the indicator of the bacterial cell amount (the value obtained by subtracting the value without the inoculation of the bacterial cells as a blank) becomes 50% or less compared to that under the condition without the addition of the culture supernatant. Moreover, the property can also be observed visually by observing a bright field image by the method described in Example 2.

Examples of Bifidobacterium adolescentis of the invention which have been obtained in the above manner from the stool of a human by the method described above include Bifidobacterium adolescentis YIT 13021, Bifidobacterium adolescentis YIT 13022, Bifidobacterium adolescentis YIT 11033, Bifidobacterium adolescentis YIT 11034 and Bifidobacterium adolescentis YIT 11040. The Bifidobacterium adolescentis of the invention includes strains which have been subjected to artificial genetic engineering or the like but which maintain the property described above, in addition to the strains listed Bifidobacterium adolescentis YIT 13021 was deposited for an international deposit as Bifidobacterium adolescentis YIT 13021 (NITE ABP-03808, accession date of Jan. 24, 2023) to an international depositary authority under the Budapest Treaty, International Patent Organism Depositary, National Institute of Technology and Evaluation (#120, 2-5-8 Kazusakamatari, Kisarazu-shi, Chiba 292-0818).

Bifidobacterium adolescentis YIT 13022 was deposited for an international deposit as Bifidobacterium adolescentis YIT 13022 (NITE ABP-03809, accession date of Jan. 24, 2023) to the international depositary authority.

Bifidobacterium adolescentis YIT 11033 was deposited for an international deposit as Bifidobacterium adolescentis YIT 11033 (NITE ABP-03810, accession date of Jan. 24, 2023) to the international depositary authority.

Bifidobacterium adolescentis YIT 11034 was deposited for an international deposit as Bifidobacterium adolescentis YIT 11034 (NITE ABP-03811, accession date of Jan. 24, 2023) to the international depositary authority.

Bifidobacterium adolescentis YIT 11040 was deposited for an international deposit as Bifidobacterium adolescentis YIT 11040 (NITE ABP-03812, accession date of Jan. 24, 2023) to the international depositary authority.

The bacteriological properties of the Bifidobacterium adolescentis of the invention described above are the same as those of the type strain of Bifidobacterium adolescentis (ATCC15703) except that the Bifidobacterium adolescentis of the invention has a function of adhering to and utilizing a starch granule and that a culture supernatant thereof has a property of suppressing adhesion of another bacterium to a starch granule.

The Bifidobacterium adolescentis of the invention explained above has the property described above and thus can be used as a strain which proliferates preferentially in the intestines using starch granules in the intestines exclusively or as a Bifidobacterium strain which exerts various health effects and the like through the proliferation.

Moreover, the Bifidobacterium adolescentis of the invention has the same properties as those of the conventionally known Bifidobacterium adolescentis except for having the property described above and thus can also be used for the same applications as those of the conventionally known Bifidobacterium adolescentis.

Furthermore, because the Bifidobacterium adolescentis of the invention is a Bifidobacterium strain, generally known advantages of bifidobacteria, such as the metabolism-improving effect through acetic acid, suppression of an allergy, protection against infection and enhancement of the effect of an anticancer agent, are also expected to be obtained.

Preferable applications of such Bifidobacterium adolescentis of the invention include drinks and foods such as fermented milk, yogurt and powdered probiotics, pharmaceuticals such as lactic acid bacteria preparations and the like. Of these applications, fermented foods such as fermented milk and pharmaceuticals such as lactic acid bacteria preparations are preferable.

Moreover, because the Bifidobacterium adolescentis of the invention has the property described above, the microbiota composition in the intestines and the production state of metabolites and the like are expected to change when the Bifidobacterium adolescentis is administered to a mammal including a human. In addition, when the Bifidobacterium adolescentis of the invention is administered to a mammal including a human together with starch granules, the effect is expected to be enhanced.

Accordingly, the Bifidobacterium adolescentis of the invention is preferably a composition combined with starch granules. Such a composition preferably contains around 10⁵ to 10¹⁰ cfu of the Bifidobacterium adolescentis of the invention as a daily dose and around 0.5 to 100 g of starch granules.

Furthermore, the culture supernatant of the Bifidobacterium adolescentis of the invention has a property of suppressing adhesion of another bacterium to a starch granule and thus can be used for isolating/removing a strain adhered to starch granules or protecting starch granules from being metabolized by a strain or for another application. Here, the culture medium for obtaining the culture supernatant is preferably a culture medium containing soluble starch, starch granules, dextrin or the like to obtain the property more easily.

### EXAMPLES

The invention is explained in detail below referring to Examples of the invention, but the invention is not limited to the Examples.

### Example 1

### Influence of Culture Supernatant of Bifidobacterium adolescentis YIT 13021 on Strain Adhering to Starch Granules (Eubacterium rectale (Strain G38)):

### <Test Method>

### (1) Used Strains

Bifidobacterium adolescentis YIT 13021 (abbreviated to as strain YIT 13021 below) which was isolated from human stool according to a general method and Eubacterium rectale strain G38 (abbreviated to as strain G38 below) which was derived from human stool and which was found to have a property of adhering to starch granules were used.

### (2) Used Starch Granules and Method for Subjecting to Test

For the test, potato starch (Bob's Red Mill, Unmodified potato starch, abbreviated to as pSt below) was used. The pst was irradiated with UV in advance in a laminar flow cabinet overnight or longer before the use. Here, the diameter of the pSt was around 10 to 120 μm under a microscope.

### (3) Preparation of Culture Supernatant of Strain YIT 13021

A culture medium obtained by dissolving semisolid GAM medium for glycolysis “Nissui” (EIKEN CHEMICAL CO., LTD., #05460, the product does not contain any saccharide source) in a predetermined amount of water, then removing the agarose contained in the culture medium by plain sedimentation and adding 1% w/v of soluble starch was used as the culture medium (sterilized with an autoclave). The culture medium was used for culturing after several days of anaerobic replacement in an anaerobic glovebox before the use.

A colony of strain YIT 13021 which was cultured on a modified GAM agar medium (containing 0.5% soluble starch as the main saccharide source) for one day was inoculated in the GAM liquid medium described above and stationary cultured at 37 °C overnight. The bacterial solution after culturing was centrifuged at 4 °C at 5,000×g, and the supernatant was collected and sterilized by filtration with Minisart (Sartorius, #RC15-AC), which is a filter having a pore size of 0.2 μm, or an equivalent filter having a diameter of 0.2 μm.

### (4) Culture of Strain G38 in Starch-Containing Culture Medium

For culturing in a starch-containing culture medium, a basal medium obtained by removing agarose from the semisolid GAM medium for glycolysis “Nissui” in the same manner as in (3) was prepared and subjected to several days of anaerobic replacement in an anaerobic glovebox before the use. Soluble starch which was separately sterilized with an autoclave and pSt which was irradiated with UV overnight or longer were added each to 0.5% w/v in an anaerobic glovebox. Furthermore, the culture supernatant of strain YIT 13021 prepared in (3) was added to 20% w/v.

A colony of strain G38 which was cultured on a modified GAM agar medium (containing 0.5% soluble starch as the main saccharide source) for one day was inoculated in the pSt-containing GAM medium described above using an inoculating loop and cultured at 37 °C for one day while shaking/rotating under an anaerobic condition with a rotator (ACR-100, shaking mode). To inactivate the protein and maintain the sample state, a part of the bacterial solution after culturing was fixed with paraformaldehyde.

### (5) Evaluation of Bacterial Cell Amount Adhered to Granules

After the completion of the culture by the method in (4), the granules were collected by the following procedures to evaluate the bacterial cell amount adhered to the pSt. After 50 μl of the bacterial culture solution fixed with paraformaldehyde was added to 1 ml of PBS, granules were precipitated by leaving still for one minute or longer, and the supernatant was discarded. PBS in a volume of 1 ml was newly added gently, and the mixture was stirred by mixing by inversion. This was left still again for one minute or longer, and the supernatant was removed. To the collected granules, 20 μl of 10 μg/ml Hoechst 33342 solution (DOJINDO LABORATORIES) was added to conduct reaction at room temperature, and the double-stranded DNA in all the bacterial cells adhered to the granules was stained. The supernatant after the reaction was removed, and after 20 μl of VECTASHIELD Mounting Medium with DAPI (VECTOR LABORATORIES, #H-1200) was added and suspended, the bacterial cells colonizing on the granules were observed with a microscope.

The average luminance value of each granule was calculated based on the Hoechst 33342 staining image, and the average luminance value was evaluated as the bacterial cell amount adhered to the granule. As a specific procedure, Hoechst 33342 staining images (magnification of 100 times) of 10 fields for each sample were read with ImageJ (ver. 1.52 g), and the regions of the granules were determined. Subsequently, the average value of the luminance in the region was calculated for each granule and was used as an indicator of the bacterial cell amount adhered to the granule.

### <Results>

### [Influence of Culture Supernatant of Strain YIT 13021 on Colonization of Strain G38 on Starch Granules]

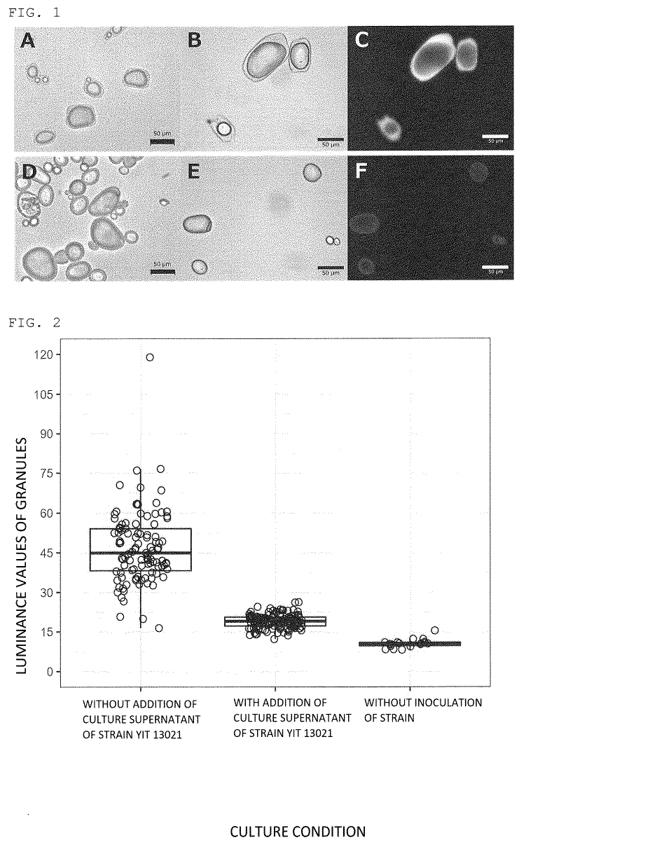

The influence of the addition of the culture supernatant of strain YIT 13021 described above during the culture on colonization of strain G38 on granules was examined. First, when the bacterial culture solution of strain G38 under the condition without the addition of the culture supernatant of strain YIT 13021 was observed with a microscope, both bacterial cells adhered to the granules and non-adherent suspending bacterial cells were observed (FIG. 1A). When the bacterial cells were observed after removing the suspending bacterial cells by washing, the colonization of strain G38 on the granules became clear (FIGS. 1B and C). On the other hand, under the condition with the addition of the culture supernatant of strain YIT 13021, although many suspending bacterial cells were observed, less bacterial cells adhered to the granules were observed (FIGS. 1D, E and F).

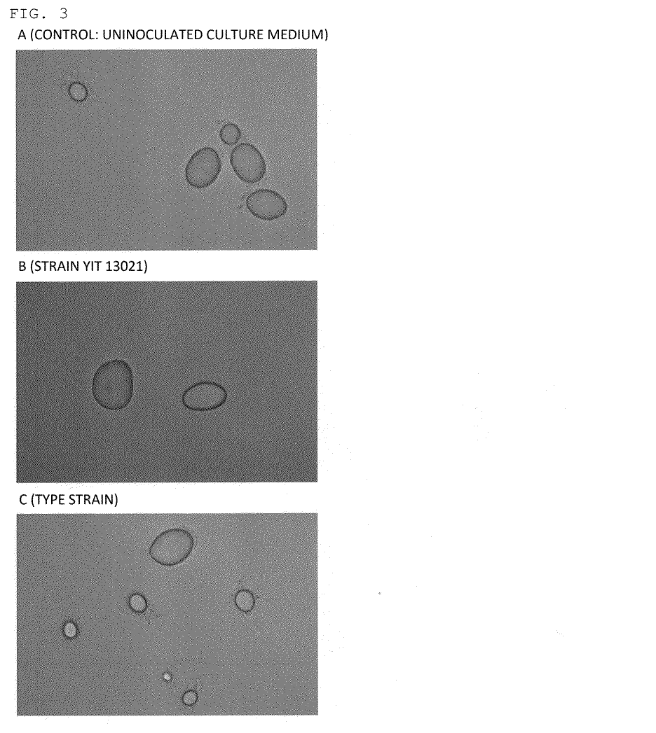

Thus, the bacterial cell amounts colonizing on the granules were calculated based on Hoechst33342 staining images and compared between the conditions. As a result, it could be observed that, although slight adhesion of the bacterial cells to the granules was observed under the condition with the addition of the culture supernatant of strain YIT 13021, the bacterial cell amount colonizing on the granules was lower than that under the condition without the addition (FIG. 2). Here, because same degrees of suspending bacterial cells which were not adhered to the granules were observed in the bright field images of the entire culture solutions under both conditions before washing the granules (FIGS. 1A and D), it was determined that the proliferation of strain G38 was not inhibited by the addition of the culture supernatant of strain YIT 13021.

### Example 2

### Influence of Culture Supernatant of Strain YIT 13021 on Strain Adhering to Starch Granules (Eubacterium rectale (Strain G38)):

### (1) Used Strains

The same strain YIT 13021 as that used in Example 1, Eubacterium rectale strain G38 and Bifidobacterium adolescentis strain YIT 4011 (abbreviated to as the type strain or strain YIT 4011 below) (ATCC15703), which is the type strain of Bifidobacterium adolescentis, were used.

### (2) Used Starch Granules and Method for Subjecting to Test

For the test, potato starch which was treated by the same method as that in Example 1 (2) was used.

### (3) Preparation of Culture Supernatants of Strain YIT 13021 and Type Strain

A culture supernatant of strain YIT 13021 was obtained by the same method as that in Example 1 (3). Moreover, a culture supernatant of the type strain was obtained by the same method as that in Example 1 (3) except for changing the cultured strain to the type strain.

### (4) Culture of Strain G38 in Starch-Containing Culture Medium and Addition of Culture Supernatant of Strain YIT 13021 or Type Strain

A culture medium obtained by dissolving semisolid GAM medium for glycolysis “Nissui” in a predetermined amount of water, then removing the agarose contained in the culture medium by plain sedimentation and adding 0.5% w/v of soluble starch and 0.5% w/v of pSt (sterilized with an autoclave, the pSt was added after sterilizing with UV and then autoclaving) was subjected to several days of anaerobic replacement in an anaerobic glovebox and then used for culturing. A colony of strain G38 which was cultured on a modified GAM agar medium (containing 0.5% soluble starch as the main saccharide source) for one day was inoculated in the pSt-containing GAM medium described above and cultured at 37 °C for one day while shaking/rotating under an anaerobic condition with a rotator (ACR-100 shaking mode).

The bacterial solution after culturing was divided into new tubes, and an equivalent amount of the uninoculated culture medium or the culture supernatant of strain YIT 13021 or the type strain prepared in (3) was added thereto. Then, the reaction was conducted at 37 °C for five hours while shaking/rotating with a rotator (ACR-100, shaking mode).

### <Results>

### [Influence of Culture Supernatant of Strain YIT 13021 or Type Strain on Colonization of Strain G38 on Starch Granules]

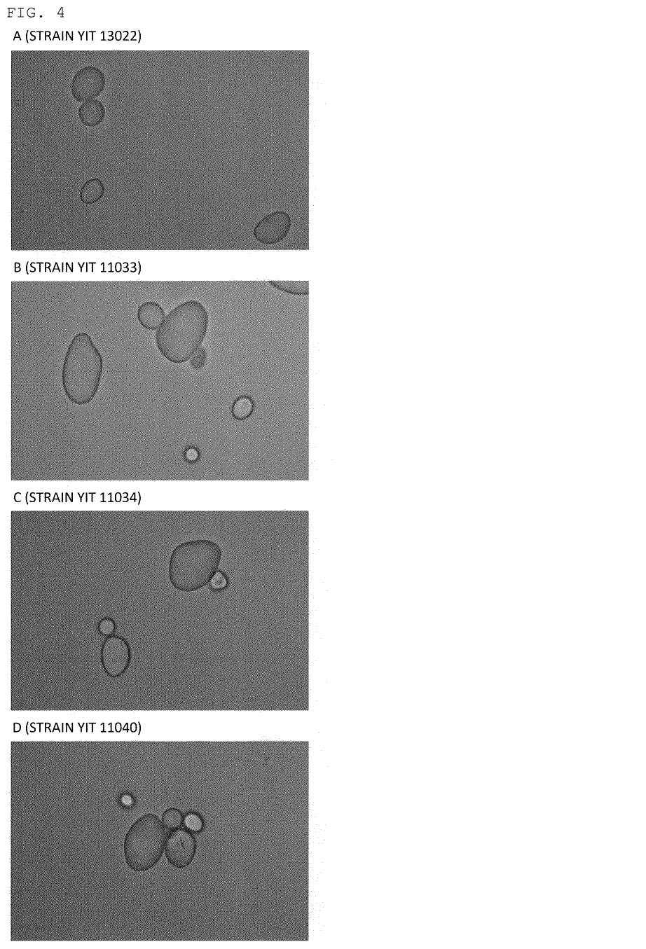

As a result of microscopic observation of the specimens, it was observed that the bacterial cells colonized on the pSt surface as those before the reaction under the condition with the addition of the uninoculated culture medium (FIG. 3A). On the other hand, it was observed that the amount of the colonizing bacterial cells decreased under the condition with the addition of the culture supernatant of strain YIT 13021 (FIG. 3B). This suggests that the culture supernatant of strain YIT 13021 suspends the pSt-colonizing bacterial cells of strain G38.

On the other hand, it was observed that the bacterial cells of strain G38 colonized on the pSt surface as those before the reaction under the condition with the addition of the culture supernatant of the type strain (FIG. 3C).

### Example 3

### Influence of Culture Supernatants of Bifidobacterium adolescentis on Strain Adhering to Starch Granules (Eubacterium rectale (Strain G38)):

The same test as that in Example 2 was conducted using Bifidobacterium adolescentis strains (other than that used in Example 1) which were isolated from human stool according to a general method.

As a result, it was found that Bifidobacterium adolescentis YIT 13022, Bifidobacterium adolescentis YIT 11033, Bifidobacterium adolescentis YIT 11034 and Bifidobacterium adolescentis YIT 11040 exert the same effect as that of Bifidobacterium adolescentis YIT 13021. (FIG. 4)

### Example 4

### Property of Adhering to and Property of Utilizing Starch Granules of Bifidobacterium adolescentis in Which Culture Supernatant has Function of Suppressing Adhesion Property of Another Strain to Starch Granules

### (1) Used Strains

Five strains, namely Bifidobacterium adolescentis YIT 13021, Bifidobacterium adolescentis YIT 13022, Bifidobacterium adolescentis YIT 11033, Bifidobacterium adolescentis YIT 11034 and Bifidobacterium adolescentis YIT 11040, were used.

### (2) Evaluation of Property of Adhering to Starch Granules

About 1 μl of colonies of the respective isolated strains which were cultured on a modified GAM agar medium (containing 0.5% soluble starch as the main saccharide source) were taken and suspended in 500 μl of PBS (bacterial suspensions). Moreover, a PBS suspension (1%, w/v) of Corn Starch (Sigma, S4126) was prepared (a starch suspension). The bacterial suspensions were mixed with the starch suspension, lightly vortexed, left still for about 30 seconds and then lightly vortexed again, and parts of the mixtures were taken and observed with a phase-contrast microscope. Thus, the properties of adhering to starch granules were evaluated. When the starch granules aggregated through crosslinking with the bacterial cells and when adhesion of the bacterial cells to the granule surfaces was observed, it was determined that the strain has a property of adhering to starch granules.

### (3) Evaluation of Property of Utilizing Starch Granules

A culture medium obtained by dissolving semisolid GAM medium for glycolysis “Nissui” in a predetermined amount of water, then removing the agarose contained in the culture medium by plain sedimentation, autoclaving and then adding 1% w/v of UV-sterilized pSt was used for culturing. After the isolated strains were inoculated and anaerobically cultured at 37 °C for three days, the pH values of the culture solutions were measured, and the strains having pH less than 5 were determined to have an utilization property.

### <Results>

As a result of microscopic observation (not shown in the figures), aggregation of the starch granules was observed in all the strains. When the relations between the individual granules and the bacterial cells were observed, adhesion of the bacterial strains to the starch granules could be observed, and the aggregates were believed to be formed as a result of crosslinking of the starch granules due to adhesion of the bacterial cells.

As a result of the pH measurement, it was found that all the strains have a property of utilizing starch granules.

From the above results, it was found that the culture supernatants of Bifidobacterium adolescentis YIT 13021, YIT 13022, YIT 11033, YIT 11034 and YIT 11040 have a property of suppressing adhesion of strain G38 to starch granules. Such property has not been known in the conventionally known Bifidobacterium adolescentis including the type strain. Furthermore, the strains themselves have a property of adhering to and utilizing starch granules and thus can use starch granules preferentially even when there is another strain having a property of adhering to starch granules. Here, it was found that, when strain YIT 13021 was inoculated to starch granules to which strain G38 was adhered, strain YIT 13021 adhered to the starch granules preferentially (data not disclosed).

### Industrial Applicability

Because the Bifidobacterium adolescentis of the invention suppresses adhesion of intestinal microbiota in competition such as Eubacterium rectale to a starch granule and adheres and colonizes itself, the Bifidobacterium adolescentis is expected to exert generally known effects of bifidobacteria which are exerted by the proliferation of Bifidobacterium adolescentis, such as the metabolism-improving effect through acetic acid, suppression of an allergy, protection against infection and enhancement of the effect of an anticancer agent and can be applied to health promotion.

1. Bifidobacterium adolescentis having a function of adhering to and utilizing a starch granule
wherein a culture supernatant thereof has a property of suppressing adhesion of another bacterium to a starch granule.

2. Bifidobacterium adolescentis YIT 13021 (NITE ABP-03808), Bifidobacterium adolescentis YIT 13022 (NITE ABP-03809), Bifidobacterium adolescentis YIT 11033 (NITE ABP-03810), Bifidobacterium adolescentis YIT 11034 (NITE ABP-03811) and Bifidobacterium adolescentis YIT 11040 (NITE ABP-03812).

3. A composition comprising the Bifidobacterium adolescentis according to claim 1 and a starch granule.

4. A culture supernatant of the Bifidobacterium adolescentis according to claim 1.

5. A composition comprising the Bifidobacterium adolescentis according to claim 2 and a starch granule.

6. A culture supernatant of the Bifidobacterium adolescentis according to claim 2.
